(19) United States
(12) Patent Application Publication
(10) Pub. No.: US 2010/0030180 A1
(43) Pub. Date: Feb. 4, 2010

(54) METHOD FOR MANUFACTURING A MULTI-LAYERED FILM AND FILM MANUFACTURED BY SAID METHOD

(75) Inventor: Johan Willy Declerck, Grimbergen (BE)

Correspondence Address: BACON & THOMAS, PLLC, 625 SLATERS LANE, FOURTH FLOOR, ALEXANDRIA, VA, 22314-1176, US

(73) Assignee: TEKNI-PLEX EUROPE, naamloze vennootschap, Erembodegem (BE)

(21) Appl. No.: 12/311,909

(22) PCT Filed: Oct. 18, 2006
(86) PCT No.: PCT/BE2006/000116

§ 371 Date: Oct. 1, 2009

(51) Int. Cl. A61B 19/00; B29C 47/06; B32B 1/02; B32B 37/06

(52) U.S. Cl. 604/408; 264/173.16; 428/35.2; 156/244.13

(57) ABSTRACT

Multi-layered film consisting of at least an inner layer (3) and an outer layer (2), with an intermediate layer (4) provided in between, whereby at least the inner and the outer layers (2-3) consist of a homopolymer of polypropylene and/or a copolymer of polypropylene, and whereby the intermediate layer (4) contains at least a mixture of a homopolymer of polypropylene and/or a copolymer of polypropylene with a thermoplastic elastomer, characterized in that the film (1) has a haze of 5% or less.

It is generally accepted that a film used for the containment of medical fluids needs to present highly transparent in order to allow visual inspection of their content.

The transparency requirement is very important as medical fluids for intravenous application need to be absolutely clear and particle free and visual inspection will allow to determine whether or not contamination has taken place and to check for the turbidity of the solution.

Other important requirements for a film used for manufacturing intravenous bags (IV-bags) for the containment of medical fluids are heat sealability, flexibility, puncture resistance, the ability to withstand sterilisation and barrier properties.

The flexibility of the film is important for the drainage of IV-bags, so that patients receive exact solution quantities as intended.

It is clear that ideally the transparency and flexibility of the film are not affected by autoclave sterilisation at high temperature, humidity and pressure, nor during low temperature storing.

Currently medical bags are typically made from films based on polyvinyl chloride (PVC). However, such PVC films are not free of low molecular weight additives, such as plasticizers, which can migrate from the IV-bag into the medical fluid contained therein. Therefore it is recommended for several applications to replace PVC by inert materials such as for instance polyolefin materials.

Alternative films have been designed and tested in industry. A known alternative film for manufacturing IV-bags consists of at least three layers, namely an outer layer and an inner layer with an intermediate layer provided in between.

The use of the terms inner and outer layer is based on the position of said layers in a finished IV-bag, more specifically, the inner layer is meant to be situated at the inside of the bag and will be in contact with the product contained therein, while the outer surface constitutes the outer surface of the IV-bag.

In said alternative film, at least the inner layer and the intermediate layer comprise a mixture of a polypropylene homopolymer or copolymer and a thermoplastic elastomer (TPE).

An inconvenience of such film, produced by a conventional method, is that the haze of such films comprising one or more layers of a mixture of polypropylene and a thermoplastic elastomer, usually around 6%, is rather high.

The goal of the present invention is to provide a solution for the above inconveniences.

Therefore the present invention concerns a multi-layered film consisting of at least an inner layer and an outer layer, with an intermediate layer provided in between, whereby at least the inner and the outer layer consist of a homopolymer of polypropylene and/or a copolymer of polypropylene, and whereby the intermediate layer contains at least a mixture of a homopolymer of polypropylene and/or a copolymer of polypropylene with a thermoplastic elastomer, characterized in that the film has a haze of 5% or less.

The present invention also relates to a method for manufacturing such a high transparent multi-layered film.

Up to now, there are known three major methods for manufacturing such a film, the first is by extrusion of the several layers and a subsequent lamination step.

Lamination however has the inconvenience that additional adhesives have to be used to laminate the different layers, which not only implies an additional production cost but which also implies that due care should be taken when selecting convenient adhesives since migration of the adhesives or residual solvents out of the film should be prevented.

A second known method is to co-extrude the layers and to subsequently chill the film in between two chill rolls.

An inconvenience is that this method cannot sufficiently prevent the growth of crystals in the film with dimensions superior to the wavelength of light, thereby reducing the transparency of the film. Also by this cooling method, the two sides of the film are unevenly cooled. This results in building in stress in the film and induces curl and shrink during thermal handling.

A third known method consists of co-extruding the layers and to subsequently quench the film in a water bath.

Although this method allows producing a very amorphous and clear film, the quenching results in a film with a somewhat wavy surface that increases the haze to around 6%.

Thereto the invention concerns a method for manufacturing a multi-layered film consisting of at least an inner layer and an outer layer, with an intermediate layer provided in between, whereby at least the inner and the outer layer consist of a homopolymer of polypropylene and/or a copolymer of polypropylene, and whereby the intermediate layer contains at least a mixture of a homopolymer of propylene and/or a copolymer of polypropylene with a thermoplastic elastomer, characterised in that the method at least comprises the steps of co-extruding said layers to create a film and subsequently chill said film between at least one rotating endless belt and a chill roll that extend complementary to each other over at least a part of their course.

Extended research and tests have shown that the method according to the invention allows to manufacture highly transparent multi-layered films comprising at least one layer containing a mixture of a homopolymer of propylene and/or a copolymer of polypropylene with a thermoplastic elastomer such as, for example, polyisobutylene (PIB) orstyrene-ethylene-butylene-styrene (SEBS), or the like.

With the intention of better showing the features of the present invention, hereafter, as an example without any limitative character, several preferred forms of embodiments are described, with reference to the accompanying drawings, wherein:

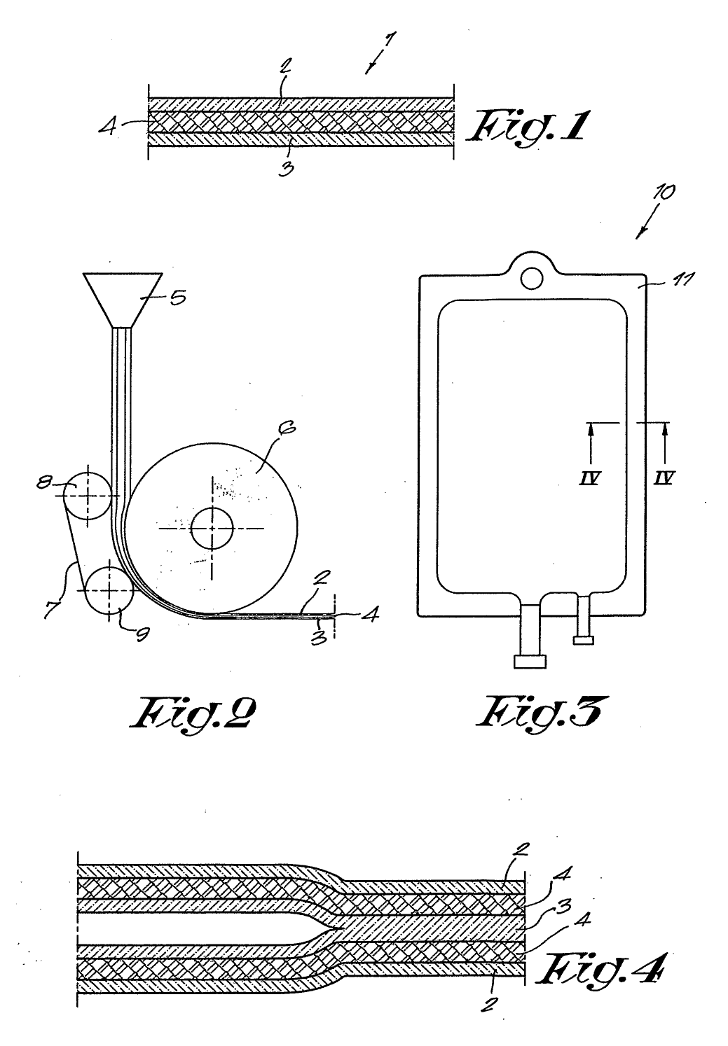

FIG. 1 represents a cross section of a film according to the invention;

FIG. 2 schematically represents a method for manufacturing a film according to the invention;

FIG. 3 represents a container manufactured with a film according to the invention;

FIG. 4 represents, on a larger scale, a cross section according to line IV-IV in FIG. 3.

FIG. 1 represents a multi-layered film 1 according to the invention consisting of at least three layers, in particular an outer layer 2, an inner layer 3 and an intermediate layer 4 provided in between the inner layer 3 and the outer layer 2.

According to the invention the outer layer 2 and the inner layer 3 both comprise a homopolymer of polypropylene and/or a copolymer of polypropylene. Both layers 2 and 3 may also comprise a thermoplastic elastomer (TPE) such as, for example, polyisobutylene, styrene-ethylene-butylene-styrene (SEBS), or the like.

The inner layer 3 preferably has a melting temperature inferior to the melting temperature of the outer layer 2, whereto the inner layer 3 preferably consists of a copolymer of polypropylene, while the outer layer preferably consists of a homopolymer of polypropylene. This type of construction enhances the heat sealing characteristics.

Also according to the invention, the intermediate layer 4 comprises a mixture of at least (a) an homopolymer of polypropylene and/or a copolymer of polypropylene, and (b) a thermoplastic elastomer (TPE), such as PIB and/or SEBS or the like.

The TPE that is used in at least the intermediate layer 4 of the film preferably is a compound containing styrene-ethylene-butylene-styrene (SEBS) next to polypropylene.

Providing TPE in the intermediate layer 4 provides a good flexibility that is required for a film used for the manufacturing of medical solution bags. The film is further autoclavable.

Preferably, no nucleating agents or low molecular weight additives, such as plasticizers, are present in the film.

The thickness of the film is typically between 100 and 300 μm, whereby the outer layer is preferably in the range of 30 μm, the thickness of the inner layer 3 is preferably in the range of 50 μm and the thickness of the intermediate layer 4 is preferably in the range of 100 μm.

The random copolymer of polypropylene can be for example Borealis® RD 204 CF, Borealis® RD 208 CF or Borealis® TD 109 CF. The homopolymer of polypropylene can be for example Borealis® DM 55 or Borealis® HD 601 CF. For a mixture of polypropylene and thermoplastic elastomer, for instance Meliflex® XS 141, Meliflex® XS 167, Meliflex® XS 140-3, Cellene® 06-121-08, Cellene® 06-121-09 or Cellene® 06-121-12 can be chosen.

According to the invention the haze of the film is less than or equal to 5%, preferably even less than 3.5% and more preferably even less than 2.5% when measured by the following standard test method: ASTM D 1003.

As represented in FIG. 2, the method for manufacturing the above film 1 at least comprises the steps of co-extruding the at least three layers 2, 3 and 4 through a die 5 in order to create a film 1. The co-extruded film 1 subsequently is chilled between at least one rotating endless belt 7 and a chill roll 6 that extend complementary to each other over at least a part of their course.

In this case the endless belt 7 consists of a metal sheet that is guided over at least two guiding rolls 8 and 9.

The roll 6 and the endless belt 7 are driven in an inverse direction and are chilled, such that the film 1 is transported in between said roll 6 and endless belt 7 and is cooled at its both surfaces during an extended period dependent on the drive speed of the roll and endless belt.

The method according to the invention offers the advantage that the chilling of the film can be performed adequately, such that the growth of crystals in the film can be prevented or at least stopped before the crystals reach dimensions equal to or greater than the wavelength of visible light, thereby resulting in a highly transparent film with a haze that is equal to or less than 5% and preferably even less than 3.5%.

In addition the polypropylene materials are carefully selected so that they cannot recrystallize at temperatures typically applied for the thermal sterilization of containers. This enables to keep the transparency very high even after autoclaving.

Essays have shown that a film according to the invention can resist an autoclave sterilisation cycle of 20 minutes at 121° C., with only a slight loss of transparency.

Indeed, films according to the invention have shown an increase in haze from an initial value of approximately 3.5% prior to the sterilisation to approximately 5% after the sterilisation.

In addition it is proven to be able to increase the polypropylene content in the outer layer 2 and inner layer 3, or even eliminate the TPE content in said layers, hence reducing the raw material cost of the co-extruded film.

The film 1 according to the present invention is especially suited for the manufacturing of medical solution bags 10.

FIGS. 3 and 4 represent a medical solution bag 10 and in particular an intravenous bag (IV bag) that is manufactured by providing two sheets of a film 1, according to the invention, with their inner layers 3 facing each other and by sealing both films 1 together at their edges 11.

It is of course also possible to fold one sheet of a film according to the invention with the inner layer 3 facing the inside and by sealing the three free edges 11 to form the desired IV bag 11.

The present invention is by no means limited to the above described embodiments, on the contrary many alternatives of the film and method according to the invention may be realised without departing from the scope of the invention.

1-11. (canceled)

12. A method for manufacturing a multi-layered film comprising at least an inner layer (3) and an outer layer (2), with an intermediate layer (4) provided in between, which comprises co-extruding layers (2, 3, and 4) to create a film (1) and subsequently chilling said film (1) between at least one rotating endless belt (7) and a chilling roller (6) that extend complementary to each other over at least a part of their course providing a haze value to the film before sterilization of 5% or less, measured according to ASTM D 1003.

13. The method according to claim 12, wherein at least the inner and the outer layers (2, 3) of the film consist of a homopolymer of polypropylene and/or a copolymer of polypropylene, and the intermediate layer (4) contains at least a mixture of a homopolymer of polypropylene and/or a copolymer of polypropylene with a thermoplastic elastomer.

14. The method according to claim 12, wherein at least the inner and the outer layers (2, 3) of the film consist of a mixture of a homopolymer of polypropylene and/or a copolymer of polypropylene with a thermoplastic elastomer, and the intermediate layer (4) contains at least a mixture of a homopolymer of polypropylene and/or a copolymer of polypropylene with a thermoplastic elastomer.

15. The method according to claim 12, wherein the film (1) has a haze value before sterilization of less than 3.5% and preferably less than 2.5%, measured according to ASTM D1003.

16. The method according to claim 12, wherein the film (1) is free of nucleating agents or low molecular weight additives.

17. The method according to claim 13, wherein the outer layer (2) of the film comprises a homopolymer of polypropylene and the inner layer (3) comprises a copolymer of polypropylene, providing the film with a melting temperature gradient.

18. The method according to claim 14, wherein the outer layer (2) of the film comprises a mixture of a homopolymer of polypropylene with a thermoplastic elastomer and that the inner layer (3) comprises a mixture of a copolymer of polypropylene with a thermoplastic elastomer, providing the film with a melting temperature gradient.

19. The method according to claim 13, wherein the thermoplastic elastomer is one selected from the group of styrene-ethylene-butylene-styrene (SEBS) and polyisobutylene (PIB).

20. The method according to claim 12, wherein the film (1) is steam sterilizable at 121 degrees Celcius for 20 minutes.

21. Medical solution bag manufactured from one or more multi-layered films produced according to the method of claim 12.

22. A method for manufacturing a medical solution bag, in particular an intravenous bag, which comprises forming a film made by the method of claim 18 into a bag shape form, heat sealing the periphery edge of the bag shape form to seal the bag and adding a medical solution to the bag.

23. A clear sterilized transparent intravenous solution bag comprising a multi-layered film comprising at least an inner layer (3) and an outer layer (2), with an intermediate layer (4) provided in between, wherein at least the inner and the outer layers (2, 3) of the film consist of a mixture of a homopolymer of polypropylene and/or a copolymer of polypropylene with a thermoplastic elastomer, and the intermediate layer (4) contains at least a mixture of a homopolymer of polypropylene and/or a copolymer of polypropylene with a thermoplastic elastomer, said bag having a haze of less than 3.5%.
